(19) United States
(12) Patent Application Publication
(10) Pub. No.: US 2012/0189663 A1
(43) Pub. Date: Jul. 26, 2012

(54) METHOD OF PRODUCING A MEDICAL COMPOSITION

(75) Inventors: Hiroshi Takaku, Chiba (JP); Tomoyuki Suzuki, Chiba (JP); Myint Oo Chang, Tokyo (JP); Norio Yamamoto, Chiba (JP); Kazuo Wakabayashi, Tottori (JP)

(73) Assignee: Chiba Institute of Technology, Narashino-shi (JP)

(21) Appl. No.: 13/261,211

(22) PCT Filed: Sep. 10, 2010
(86) PCT No.: PCT/JP2010/065628

§ 371 Date: Apr. 16, 2012

(30) Foreign Application Priority Data
Sep. 10, 2009 (JP) 2009-209318

(51) Int. Cl. A61K 39/00; A61P 37/04; G01N 33/569; A61P 35/00; C12N 5/078; C12Q 1/70

(52) U.S. Cl. 424/277.1; 435/375; 435/5

(57) ABSTRACT

The present invention aims to produce a safe and reliable medical composition, which efficiently boosts nonspecific immunity of antigen-presenting cells and thereby promotes an antitumor activity. To produce the medical composition, antigen-presenting cells such as dendritic cells are exposed to an activating reagent containing baculoviruses. Then, the antigen-presenting cells are separated from the activating reagent. The antigen-presenting cells are optionally cultured after the separation. Furthermore, an absence of the baculoviruses in the composition are optionally checked. The medical composition produced by the present invention is expected to have an outstanding therapeutic effect.

This application is a national phase application under 35 U.S.C. §371 of International Application Serial No. PCT/JP2010/065628, filed on Sep. 10, 2010, and claims the priority under 35 U.S.C. §119 to Japan Patent Application No. 2009-209318, filed on Sep. 10, 2009. These applications are hereby incorporated by reference.

### TECHNICAL FIELD

The present invention relates to a method of producing a medical composition containing antigen-presenting cells having an antitumor activity as an active ingredient, using a baculovirus.

### BACKGROUND OF THE INVENTION

No effective cancer treatments have been established yet, and new treatments are still actively being developed. Immune cell therapy is drawing attention as a new alternative therapy instead of chemotherapy using anticancer drugs which is the current mainstream of cancer therapies. A dendritic cell therapy is one type of immune cell therapy. It is a technique to manipulate dendritic cells obtained from a cancer patient in vitro. Then, the manipulated dendritic cells are infused to the patient.

Patent documents 1, 2 and 3 disclose techniques to treat dendritic cells with a cancer antigen peptide or with a nucleic acid encoding a cancer antigen. Thereby, the specific immunity of dendritic cells is enhanced.

Methods using viruses have also been studied to enhance the specific immunity of dendritic cells. Patent document 4 discloses a method of transducing dendritic cells, using an adenovirus in vitro. Patent document 5 discloses a method of introducing an antigen to dendritic cells, using a low-virulent herpes virus in vitro.

Patent documents 6, 7, 8 and 9 disclose a method of activating or maturing dendritic cells, using CpG to enhance the nonspecific immunity of dendritic cells. Patent documents 6, 10, 11 and 12 disclose a method of activating or maturing dendritic cells, using LPS to enhance the nonspecific immunity of dendritic cells.

Therapeutic agents for cancers using baculovirus have also been developed. Recently, it was discovered that the baculovirus can induce an immune response. Based on this discovery, preventive and therapeutic agents containing baculovirus have been developed for liver failure (Patent document 13).

### Prior Art Documents

### Patent Documents

Patent document 1: Japanese Patent Application Publication 2009-137857

Patent document 2: Japanese Patent Application Publication 2008-119004

Patent document 3: Japanese Patent Application Publication 2006-280324

Patent document 4: Japanese Patent Application Publication (Translation of PCT Application) 2005-523942

Patent document 5: Japanese Patent Application Publication (Translation of PCT Application) 2003-502008

Patent document 6: Japanese Patent Application Publication (Translation of PCT Application) 2009-519234

Patent document 7: Japanese Patent Application Publication (Translation of PCT Application) 2007-501607

Patent document 8: Japanese Patent Application Publication (Translation of PCT Application) 2006-510667

Patent document 9: Japanese Patent Application Publication (Translation of PCT Application) 2005-528899

Patent document 10: Japanese Patent Application Publication 2007-312683

Patent document 11: Japanese Patent Application Publication 2004-298181

Patent document 12: Japanese Patent Application Publication (Translation of PCT Application) 2004-531496

### SUMMARY OF THE INVENTION

### Problem to be Solved by the Invention

Although specific immunities were boosted by the cancer antigens or viruses, expected therapeutic effects were not often achieved by the traditional dendritic cell therapies. Therefore, it was considered impossible to establish an effective therapy just by enhancing the specific immunity of the dendritic cells. However, even when the nonspecific immunity of the dendritic cells was boosted by LPS or CpG, the antitumor activities of the dendritic cells were insufficient.

The therapeutic agent containing the baculovirus disclosed in Patent document 13 was directly administered to the patient. Since a large amount of baculovirus was necessary, it was impossible to efficiently induce an antitumor activity by this method. In addition, there was also a possibility that the patient or his family would feel anxious about the active virus being directly injected into the patient.

The present invention intends to produce a safer and more reliable medical composition, which demonstrates a remarkable therapeutic effect on antitumor activity that is acquired by effectively boosting the nonspecific immunity of antigen-presenting cells such as dendritic cells.

### Means to Solve the Problem

The present invention provides a method of producing a medical composition containing antigen-presenting cells having antitumor activity as an active ingredient. This method includes the steps of exposing the antigen-presenting cells to an activating reagent containing baculoviruses, and separating the antigen-presenting cells from the activating reagent.

According to the present invention, the baculoviruses present in the activating reagent are separated or degraded. Thus, the baculoviruses do not substantially exist in the medical composition produced. In addition, the medical composition obtained by the method of the present invention has a high antitumor activity.

The method may further include a step of culturing the antigen-presenting cell after separating the antigen-presenting cells from the activating reagent. This step further boosts the antitumor activity of the antigen-presenting cells.

Furthermore, the method may also include a step of confirming an absence of the baculoviruses in a solution containing the antigen-presenting cells after separating the antigen-presenting cells from the activating reagent. Since this step enables verifying that the baculoviruses are not present in the medical composition produced, safety and reliability of the medical composition produced by the present invention are enhanced.

An activating reagent containing approximately 50 pfu of baculoviruses per antigen-presenting cell can be used to expose the antigen-presenting cells to the activating reagent. This amount of baculoviruses is optimal in that a large number of baculoviruses are incorporated into the antigen-presenting cell and in that viability of the antigen-presenting cells is well maintained.

The antigen-presenting cells can be obtained from a cancer patient. If the produced medical composition is administered back to the cancer patient, no rejection will occur because the antigen-presenting cells originate from the cancer patient himself.

Furthermore, dendritic cells can be used as the antigen-presenting cells.

### Effect of the Invention

According to the present invention, it is possible to produce a medical composition containing antigen-presenting cells having an antitumor activity with enhanced nonspecific immunity. The medical composition produced by the present invention has a higher antitumor activity than the medical compositions containing traditional antigen-presenting cells. Therefore, the medical composition produced by the present invention is expected to have a significant therapeutic effect.

Since the medical composition produced by the present invention does not contain baculoviruses, it is safer and more reliable. Thus, it will be easier to obtain consent from the patient or his family in order to administer the medical composition of the present invention. Furthermore, since the baculoviruses are incorporated into the antigen-presenting cells in vitro, a smaller amount of baculoviruses is used for the present invention than the traditional method in which therapeutic agents are directly administered to the patient.

### BRIEF DESCRIPTION OF THE DRAWINGS

FIG. 1 is a micrograph of immunofluorescent staining, showing incorporation of baculoviruses into dendritic cells.

FIG. 2 is a photograph of electrophoresis on PCR products, showing the amounts of baculoviruses incorporated into dendritic cells after the dendritic cells were exposed to the activating reagent, in which amounts of the baculoviruses were varied.

FIG. 3 is a graph showing the viability of dendritic cells exposed to an activating reagent, in which amounts of the baculovirus were varied.

FIG. 4 is a photograph of electrophoresis on PCR products, showing the degradation of baculoviruses incorporated into dendritic cells.

FIG. 5 is a graph showing the changes of expressions of surface molecules in baculovirus—incorporated dendritic cells. ‘N.C.’ is untreated dendritic cells, ‘BV’ is the dendritic cells exposed to baculovirus—containing activating reagent, ‘LPS’ is the dendritic cells exposed to LPS—containing activating reagent, and ‘CpG’ is the dendritic cells exposed to CpG-ODN—containing activating reagent.

FIG. 6 is a graph showing the production of IFN-α by baculovirus—incorporated dendritic cells. ‘N.C.’ is untreated dendritic cells, ‘BV’ is the dendritic cells exposed to baculovirus—containing activating reagent, ‘LPS’ is the dendritic cells exposed to LPS—containing activating reagent, and ‘CpG’ is the dendritic cells exposed to CpG-ODN—containing activating reagent.

FIG. 7 is a graph showing the production of IFN-γ by baculovirus—incorporated dendritic cells. ‘N.C.’ is untreated dendritic cells, ‘BV’ is the dendritic cells exposed to baculovirus—containing activating reagent, ‘LPS’ is the dendritic cells exposed to LPS—containing activating reagent, and ‘CpG’ is the dendritic cells exposed to CpG-ODN—containing activating reagent.

FIG. 8 is a graph showing the production of TNF-α by baculovirus—incorporated dendritic cells. ‘N.C.’ is untreated dendritic cells, ‘BV’ is the dendritic cells exposed to baculovirus-containing activating reagent, ‘LPS’ is the dendritic cells exposed to LPS-containing activating reagent, and ‘CpG’ is the dendritic cells exposed to CpG-ODN-containing activating reagent.

FIG. 9 is a graph showing the production of IL-6 by baculovirus-incorporated dendritic cells. ‘N.C.’ is untreated dendritic cells, ‘BV’ is the dendritic cells exposed to baculovirus-containing activating reagent, ‘LPS’ is the dendritic cells exposed to LPS-containing activating reagent, and ‘CpG’ is the dendritic cells exposed to CpG-ODN—containing activating reagent.

FIG. 10 is a graph showing the production of IL-10 by baculovirus—incorporated dendritic cells. ‘N.C.’ is untreated dendritic cells, ‘BV’ is the dendritic cells exposed to baculovirus-containing activating reagent, ‘LPS’ is the dendritic cells exposed to LPS—containing activating reagent, and ‘CpG’ is the dendritic cells exposed to CpG-ODN—containing activating reagent.

FIG. 11 is a graph showing the production of IL-12p70 by baculovirus—incorporated dendritic cells. ‘N.C.’ is untreated dendritic cells, ‘BV’ is the dendritic cells exposed to baculovirus-containing activating reagent, ‘LPS’ is the dendritic cells exposed to LPS-containing activating reagent, and ‘CpG’ is the dendritic cells exposed to CpG-ODN-containing activating reagent.

FIG. 12 is a graph showing the expression of CD69 after co-culturing NK cells with baculovirus-incorporated dendritic cells. ‘N.C.’ is the expression of CD69 in NK cells without being co-cultured with the dendritic cells, ‘Control’ is the expression of CD69 in NK cells co-cultured with untreated dendritic cells, and ‘BV’ is the expression of CD69 in NK cells co-cultured with baculovirus-incorporated dendritic cells.

FIG. 13 is a graph showing the expression of CD69 after co-culturing CD4+ T cells with baculovirus-incorporated dendritic cells. ‘N.C.’ is the expression of CD69 in CD4+ T cells without being co-cultured with the dendritic cells, ‘Control’ is the expression of CD69 in CD4+ T cells co-cultured with untreated dendritic cells, and ‘BV’ is the expression of CD69 in CD4+ T cells co-cultured with baculovirus—incorporated dendritic cells.

FIG. 14 is a graph showing the expression of CD69 after co-culturing CD8+ T cells with baculovirus-incorporated dendritic cells. ‘N.C.’ is the expression of CD69 in CD8+ T cells without being co-cultured with the dendritic cells, ‘Control’ is the expression of CD69 in CD8+ T cells co-cultured with untreated dendritic cells, and ‘BV’ is the expression of CD69 in CD8+ T cells co-cultured with baculovirus—incorporated dendritic cells.

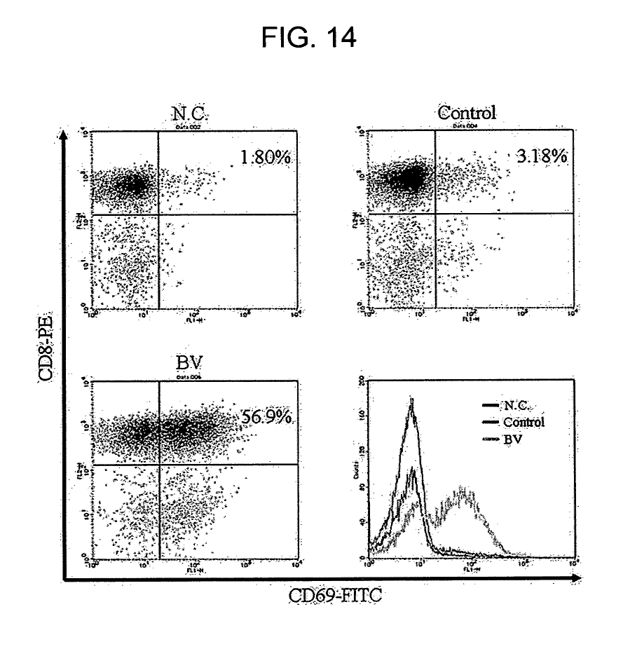

FIG. 15 is a graph showing the production of IFN-γ after co-culturing NK cells with baculovirus-incorporated dendritic cells.

FIG. 16 is a graph showing the production of IFN-γ after co-culturing CD4+ T cells with baculovirus-incorporated dendritic cells.

FIG. 17 is a graph showing the production of IFN-γ after co-culturing CD8+ T cells with baculovirus-incorporated dendritic cells.

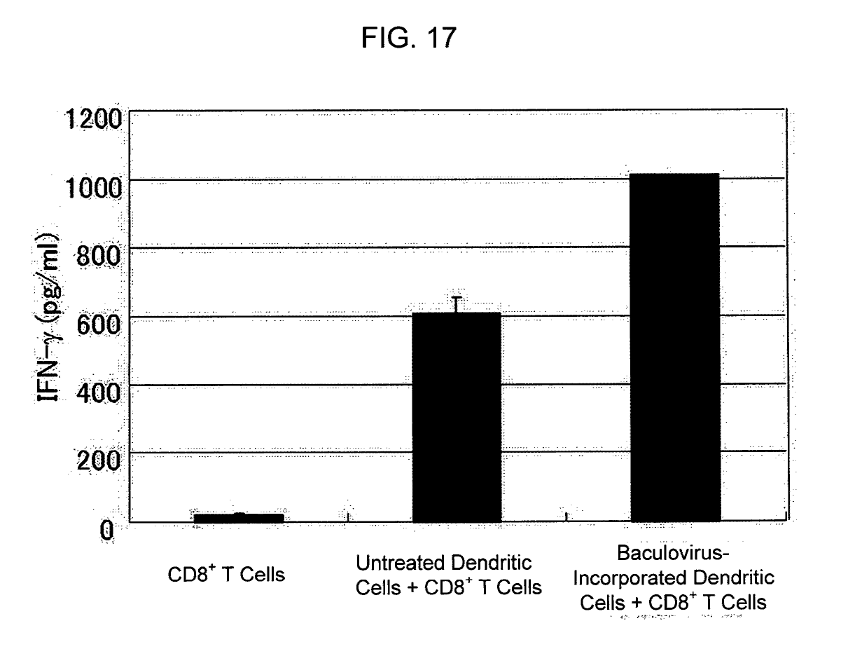

FIG. 18 is a graph showing the cytotoxic activity of NK cells after co-culturing with baculovirus-incorporated dendritic cells.

FIG. 19 is a graph showing the cell growth of CD4+ T cells co-cultured with baculovirus—incorporated dendritic cells.

FIG. 20 is a graph showing the cell growth of CD8+ T cells co-cultured with baculovirus—incorporated dendritic cells.

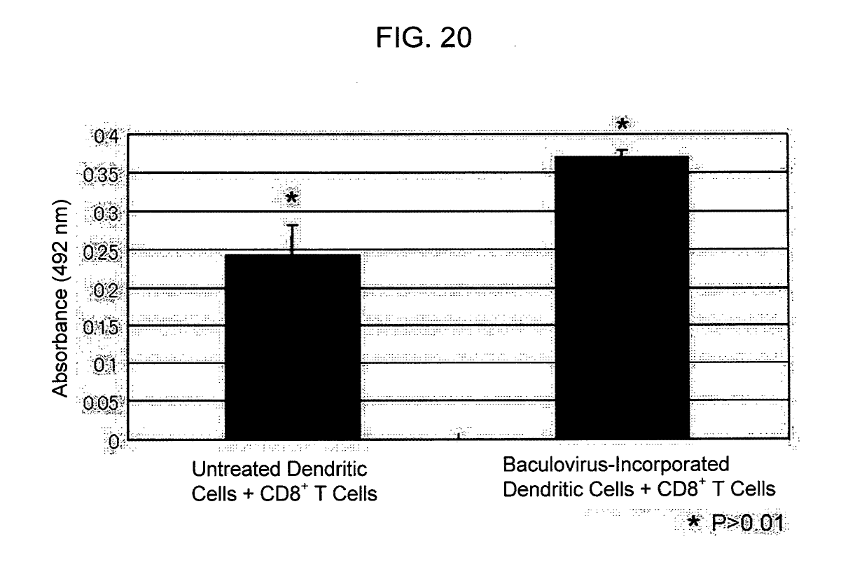

FIG. 21 is a graph showing the expression of CD69 by NK cells in spleen after administering the medical composition produced by the method of the present invention.

FIG. 22 is a graph showing the expression of CD69 by CD4+ T cells in spleen after administering the medical composition produced by the method of the present invention.

FIG. 23 is a graph showing the expression of CD69 by CD8+ T cells in spleen after administering the medical composition produced by the method of the present invention.

FIG. 24 is a graph showing the production of IFN-γ in serum after administering the medical composition produced by the method of the present invention.

FIG. 25 is a graph showing the cytotoxic activity of NK cells in spleen after administering the medical composition produced by the method of the present invention.

FIG. 26 is photographs showing that a lung cancer mouse model was generated by administering a lung cancer cell line (LLC) into mice.

FIG. 27 is a graph showing the number of lung nodules in the mouse to which the medical composition produced by the method of the present invention was administered.

FIG. 28 is photographs showing the morphology of the lung after administering the medical composition produced by the method of the present invention to a lung cancer mouse model. Example 1, Comparative Example 1 and Comparative Example 3 show the morphologies of lungs of the mice to which medical compositions of respective examples were administered once.

FIG. 29 is photographs showing HE staining of lung tissues after administering the medical composition produced by the method of the present invention to a lung cancer mouse model. Example 1, Comparative Example 1, and Comparative Example 3 show the morphologies of lungs of the mice to which medical compositions of respective examples were administered once. Asterisk indicates necrosis in the tumor, arrow indicates hemorrhage, and arrowhead indicates tumor formation.

FIG. 30 is a graph showing a survival rate of the mice to which the medical composition produced by the method of the present invention was administered.

### DETAILED DESCRIPTION OF THE INVENTION

The present invention provides a method of manufacturing a medical composition containing antigen-presenting cells having an antitumor activity as an active ingredient. This method includes an exposure step of exposing antigen-presenting cells to an activating reagent containing baculoviruses, and a separation step of separating the antigen-presenting cells from the activating reagent.

Below, preferable embodiments of the exposure step and the separation step are described to produce the medical composition. However, the present invention is not limited to only these embodiments.

(1) Exposure Step

In this step, antigen-presenting cells are exposed to an activating reagent containing baculoviruses. Because the baculoviruses in the activating reagent are incorporated into the antigen-presenting cells, the antigen-presenting cells are activated.

The antigen-presenting cells are the cells that present the fragments of proteins (derived from, for example bacteria, viruses, etc. invading the body) on their own surfaces and that activate other immune cells. The antigen-presenting cells include dendritic cells, monocytes, macrophages and B cells. The dendritic cells produce cytokines such as interferon, and play a very important role in the immune system because they activate NK cells, T cells, NKT cells and B cells.

The antigen-presenting cells can be obtained from bone marrow or blood by standard methods. To obtain the dendritic cells from blood, white blood cells in the blood are cultured in the presence of proteins such as GM-CSF and IL-4. To obtain the dendritic cells from bone marrow, bone marrow progenitor cells in the bone marrow aspirate are cultured in the same manner. The cultured cells are labeled with an antibody that binds to a protein, which is specifically expressed on the surface of the dendritic cell. Then, the dendritic cells are isolated by standard methods such as magnetic separation.

The antigen-presenting cells acquire an antitumor activity by incorporating the baculoviruses contained in the activating reagent. It is considered that the activated antigen-presenting cells activate NK cells and T cells by secreting various cytokines in the body. Then, the activated NK cells and T cells are considered to attack tumor cells.

It is preferable to use the antigen-presenting cells obtained from the patient or animal, to which the medical composition of the present invention is planned to be administered. By using the patient's own cells, rejection is prevented after the administration of the medical composition. Even in the case where the medical composition contains antigen-presenting cells obtained from a third person, it is highly likely that the rejection is avoided if the HLA antigen of the patient or animal matches with that of the third person.

The baculovirus is a virus pathogenic to insects and contains circular double-stranded DNA (molecular weight 59×10⁶−164×10⁶) as its gene. Examples of the baculoviruses known include Nucleopolyhedrovirus (NPV), geanulovirus (GV), and non-occluded virus. Examples of the Nucleopolyhedroviruses include Autographa carifornica nuclear polyhedrosis virus (AcNPV) and Bombyx mori nuclear polyhedrosis virus (BmNPV).

The baculoviruses have a very high host-specificity. The baculoviruses are unable to replicate in mammalian cells, and DNAs of the baculoviruses are not integrated into mammalian chromosomes. Therefore, the baculoviruses are extremely safe for mammals. Thus, even if the antigen-presenting cells incorporate the baculoviruses, it is considered that the viability of the cell is barely affected.

The activating reagent used in this step is a liquid containing at least the baculoviruses. By exposing the antigen-presenting cells to the activating reagent, the antigen-presenting cells incorporate the baculoviruses and become activated. As a result, the nonspecific immunity of the antigen-presenting cells is boosted, and the antigen-presenting cells acquire antitumor activity.

It is preferable that the activating reagent does not contain foreign substances other than the baculoviruses. If those impurities exist, the antigen-presenting cells cannot efficiently incorporate the baculoviruses. However, the activating reagent is preferably isotonic to the antigen-presenting cells. If the activating reagent is not isotonic, the activating reagent might cause the antigen-presenting cells to expand or shrink due to the osmotic pressure. It might adversely affect the function of the antigen-presenting cells. Therefore, the activating reagent can be physiological saline solution, phosphate buffer or serum-free cell-culture medium, in which the baculoviruses are dispersed.

The baculoviruses contained in the activating reagent is preferably approximately 1-100 pfu, and most preferably approximately 50 pfu per antigen-presenting cell. If the amount of the baculoviruses contained in the activating reagent is small, the antitumor activity the antigen-presenting cells acquire might be insufficient. If the amount of the baculoviruses is large, the manufacturing cost of the medical composition will unnecessarily increase.

The antigen-presenting cells can be exposed to the activating reagent by mixing the antigen-presenting cells and the activating reagent. An efficient exposure is achieved by occasionally stirring the mixture during the exposure. Although the exposure time is not particularly limited, the exposure time is preferably 30 minutes to 2 hours, and most preferably 1 hour. If the exposure time is too short, uptake of the baculoviruses by the antigen-presenting cells might be insufficient. If the exposure time is too long, the viability of the antigen-presenting cells might be lowered.

(2) Separation Step

After the exposure step (1), the antigen-presenting cells and the activating reagent are separated from each other. By this step, the baculoviruses that were not incorporated into the antigen-presenting cells are removed.

Centrifugation can be used to separate the antigen-presenting cells from the activating reagent. By centrifuging the mixture of the antigen-presenting cells and the activating reagent in a condition in which the antigen-presenting cells are precipitated but the baculoviruses are not precipitated, the antigen-presenting cells are separated from the activating reagent. It is preferable to centrifuge the mixture at 100 G to 300 G, and most preferably at 200 G.

Furthermore, it is preferable to centrifuge the mixture for 1 minute to 10 minutes, and more preferably for 3 minutes to 5 minutes. If the duration of centrifuge is too short, the separation of the antigen-presenting cells from the activating reagent might be insufficient. If the duration of centrifuge is too long, the viability of the antigen-presenting cells might be reduced.

Moreover, it is preferable to wash the antigen-presenting cells after separating the antigen-presenting cells. This makes the removal of the baculoviruses surer. Washing can be done by suspending the antigen-presenting cells in a suspending solution, precipitating the antigen-presenting cells by centrifuge, and removing the supernatant. A solution that does not affect the function of the antigen-presenting cells is used for the suspending solution such as cell-culture medium, physiological saline solution and phosphate buffer solution.

In the above description, the embodiments of exposure step (1) and separation step (2) were explained. Other than these steps, in the present invention, a culturing step and a confirmation step may be performed. Below, embodiments of the culturing step and the confirmation step are explained. However, the present invention is not limited to these embodiments.

### (3) Culturing Step

In this step, the antigen-presenting cells obtained by the separation step (2) are cultured. By culturing the activated antigen-presenting cells, it is possible to further boost the antitumor activity of the antigen-presenting cells.

Well-known media can be used for culturing the antigen-presenting cells. The duration period of culturing is preferably 1 hour to 48 hours. The culturing temperature is preferably 30° C. to 40° C., and most preferably 37° C.

The baculoviruses that were not separated by the separation step (2) are degraded during this step. Thus, the baculoviruses are not substantially contained in the medical composition produced by the present invention.

### (4) Confirmation Step

This step is to verify the absence of baculoviruses in the composition containing the antigen-presenting cells obtained after the separation step (2). When the culturing step (3) is performed, this step may be performed after the culturing step (3) or between the separation step (2) and the culturing step (3). By conducting this step, the absence of the baculoviruses is proved in the medical composition produced by the method of the present invention.

Examples of the methods of checking the absence of the baculoviruses are detecting a baculovirus-specific protein or DNA, and mixing the composition and insect cells and culturing the insect cells. Since the results are obtained quickly, detecting the baculovirus-specific protein or DNA is preferable.

Immunostaining can be used to detect a baculoviral protein. PCR can be used to detect a baculoviral DNA. Due to its high sensitivity of detection and easy operation, PCR is preferable.

If by chance the baculoviruses are detected, the antigen-presenting cells can be washed again to eliminate the baculoviruses. By repeating washing until the absence of the baculoviruses is confirmed, a safe and reliable medical composition can be produced.

In the above description, the embodiments of culturing step (3) and confirmation step (4) were explained.

The medical composition produced by the present invention contains antigen-presenting cells having antitumor activity. The medical composition may also contain carriers or diluents that are pharmaceutically or veterinarily acceptable. The medical composition of the present invention can be administered to species such as human, dog, cat, monkey, mouse and rat that have an immune system having antigen-presenting cells. Although the administration route is not specifically limited, an intravenous or intratumoral administration is preferred.

The medical composition produced by the present invention can be used for the treatment of a cancer or malignant tumor such as lung cancer, liver cancer, stomach cancer, colon cancer, kidney cancer, brain tumor, cervical cancer, breast cancer and bile duct cancer.

### EXAMPLES

The present invention is explained in more detail based on examples.

However, the present invention is not limited to these examples. In the below examples, mice were employed as an animal model. However, it is evident that the same practices can be applied to other animals that have immune systems similar to mice such as human, dog, cat, monkey and rat.

Preparation of Dendritic Cells

Bone marrow cells were harvested from the extremities of mice (C57BL/6, 6-8 weeks old, female), which had been sacrificed by chloroform. Hemolysis was performed using RBC lysis solution (1.54 mol/L NH4Cl, 14 mmol/L NaHCO₃, 0.1 mmol/L EDTA2Na, pH 7.3). The bone marrow cells were cultured for 7 days using an RPMI medium (Sigma-Aldrich) containing 10% FBS (Fetal Bovine Serum), 2 mmol/L L-glutamine, 2 μmol/L 2-mercaptoethanol, 20 ng/mL mouse GM-CSF and 20 ng/mL mouse IL-4. The medium was replaced by a new medium on day 3 and day 5. On day 7, the cultured cells were labeled with mouse CD11c microbeads (Miltenyi Biotec), and dendritic cells expressing CD11c+ were isolated using a MidiMACS™ separator (Miltenyi Biotec). It was confirmed by FACS that more than 90% of the population in the isolated cells was dendritic cells.

(1) Exposure Step

The dendritic cells were transferred to a 1.5 mL tube. Then, 100 μL of an activating reagent was added to the tube. The activating reagent was prepared by dispersing wild-type baculoviruses in a physiological saline solution. The dendritic cells were exposed by the activating reagent for 1 hour in an incubator set at 37° C. and containing 5% CO₂. The tube was tapped every 15 minutes to stir.

(2) Separation Step

The RPMI medium containing 2% FBS (Sigma-Aldrich) was added in the 1.5 mL tube to adjust the volume to be 1 mL. The tube was centrifuged at 200 G for 4 minutes at 4° C., and the dendritic cells were precipitated. The supernatant containing the activating reagent was discarded, and the dendritic cells were separated from the activating reagent. Then, the dendritic cells were suspended in 1 mL of RPMI medium described above. The suspension solution was centrifuged at 200 G for 4 minutes at 4° C., and the supernatant was discarded. Thereby, the dendritic cells were washed.

(3) Culturing Step

The dendritic cells were cultured for 3-48 hours in the RPMI culture medium (Sigma-Aldrich). After incubating, the medium was centrifuged at 200 G for 4 minutes at 4° C., and the RPMI culture medium was discarded. The dendritic cells were suspended in 1 mL of physiological saline solution. The suspension solution was centrifuged at 200 G for 4 minutes at 4° C., and the supernatant was discarded. Thereby, the dendritic cells were washed. Then, the dendritic cells were again suspended in 1 mL of physiological saline solution. Thereby, the medical composition was obtained.

(4) Confirmation Step

An aliquot of the medical composition was collected, and the dendritic cells were precipitated by centrifuging at 1500 rpm for 4 minutes at 4° C. PCR was performed on the supernatant, using gp64-specific primer sets. The PCR product was analyzed by 1.5% agarose gel electrophoresis. Since no band was detected, it was confirmed that the baculoviruses were not contained in the medical composition produced.

Below, properties of the medical composition produced were studied.

<<1. Verification of Incorporation of Baculoviruses into Dendritic Cells>>

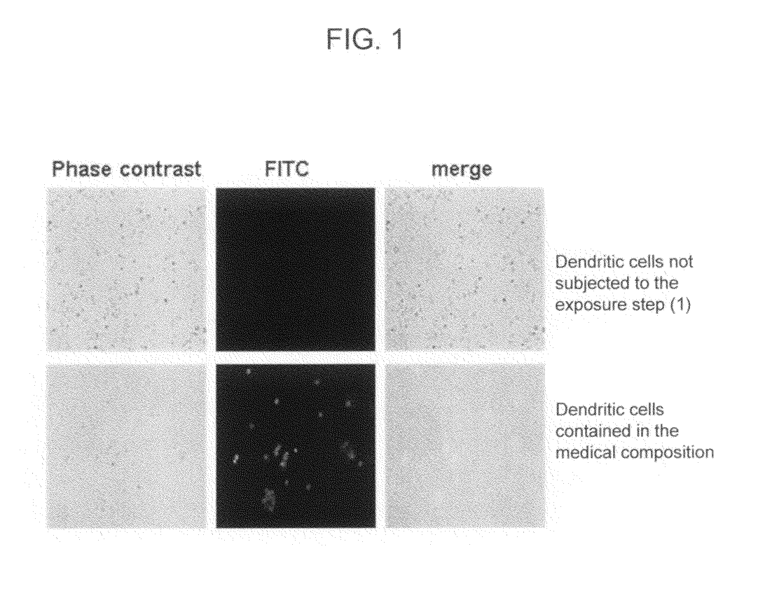

After conducting the culturing step (3), the dendritic cells contained in the medical composition were collected by centrifugation. The cells were fixed with 2% paraformaldehyde, and permeabilized with 0.2% Tween20/PBS. Dendritic cells that were not subjected to the exposure step (1) were used as a control. The cells were stained with mouse anti-gp64 IgG and goat anti-mouse IgG-FITC. Then, the envelope protein gp64 of the baculovirus in the dendritic cells was observed under fluorescence microscope. As shown in FIG. 1, the presence of gp64 was confirmed in the dendritic cells contained in the medical composition. Thus, it was confirmed that the dendritic cells had incorporated the baculoviruses in vitro by the method of present invention.

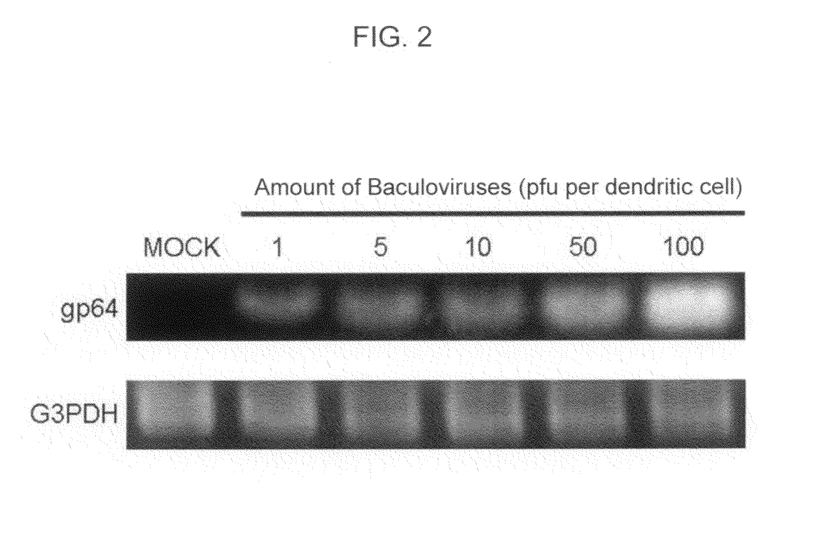

The exposure step (1) was carried out, varying the amount of baculoviruses contained in the activating reagent. The dendritic cells were exposed to activating reagents containing 0, 1, 5, 10, 50 and 100 pfu of baculoviruses per dendritic cell respectively. 4 hours later, whole DNA in the dendritic cells was isolated. Then, PCR was performed, using gp64—specific primer sets, and the PCR products were analyzed by 1.5% agarose gel electrophoresis. As shown in FIG. 2, the amounts of baculoviruses taken up by the dendritic cells correlated to the amounts of baculoviruses contained in the activating reagents. Incorporation of the baculoviruses was confirmed even with 1 pfu of baculoviruses per dendritic cell. It was further confirmed that the incorporation of the baculoviruses was highly efficient when the activating reagent contained 50 pfu of the baculoviruses per dendritic cell.

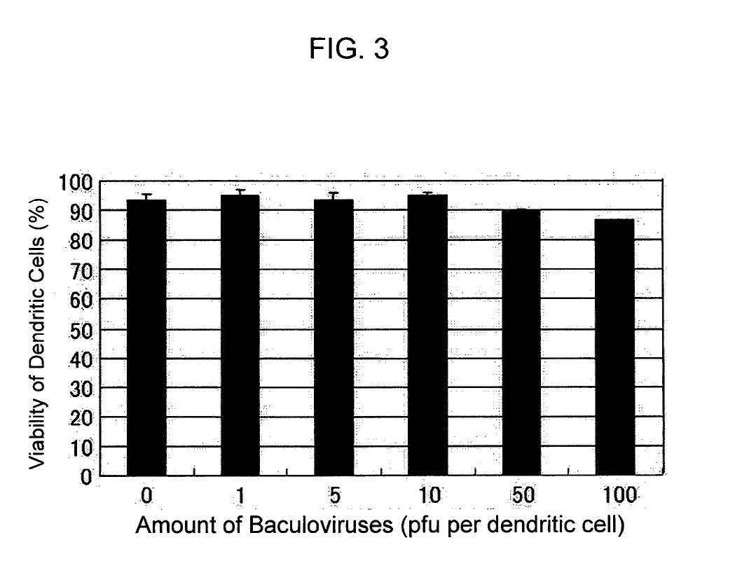

24 hours after the exposure step (1), the dendritic cells were harvested and stained with trypan blue. Then, the viability rate of the dendritic cells was calculated. As shown in FIG. 3, the viability rate of the dendritic cells was more than 85% even after a large amount of baculoviruses were incorporated into the dendritic cells. Therefore, a high safety of the baculovirus was confirmed.

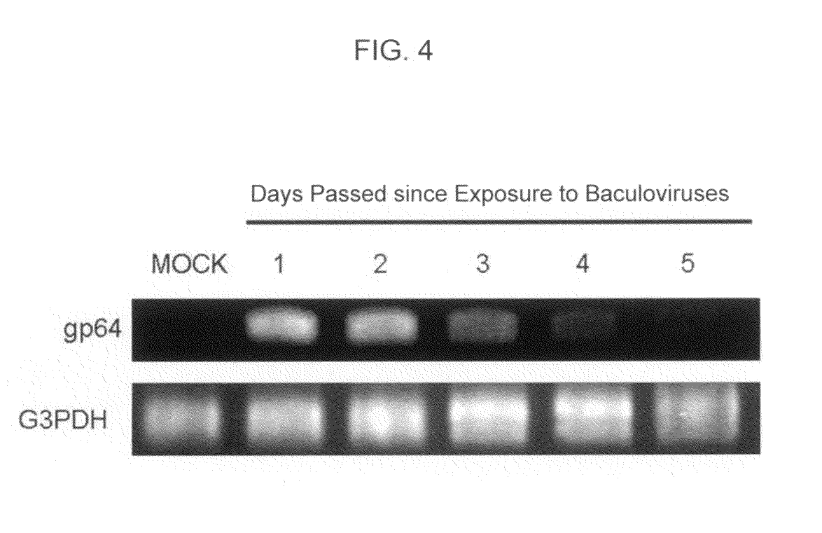

During exposure step (1), the dendritic cells were exposed to the activating reagent containing 50 pfu of baculoviruses per dendritic cell. In the culturing step (2), the dendritic cells were cultured for 6 days, and whole DNA of the dendritic cells was collected every day. PCR was performed using gp64-specific primer sets, and the PCR products were analyzed by 1.5% agarose gel electrophoresis. As shown in FIG. 4, after the dendritic cells uptook the baculovirus, the viruses began to be degraded on day 2 and were completely degraded by day 5. It was found that baculoviral DNA was degraded without being incorporated into the chromosomes of the cells. Therefore, a high safety of the baculovirus was again confirmed.

<<2. Verification of Nonspecific Immunity of Baculovirus-Incorporated Dendritic Cells>>

Dendritic cells uptake the foreign bacteria and viruses that are invading the body, bind the fragments of antigen to MHC molecules, and present them to T cells. To do this, binding of CD40, CD80 and CD86, surface molecules (co-stimulator molecules) of the dendritic cell, to CD40L and CD28, corresponding ligands of the T cells, is essential. It is also known that IFN-α and IL-12, cytokines produced by the dendritic cells, strongly activate NK cells and T cells. Therefore, expression of MHC molecules and costimulator molecules as well as production of cytokines were measured as an indicator of the boost of nonspecific immunity caused by the dendritic cells that were activated by the uptake of baculoviruses.

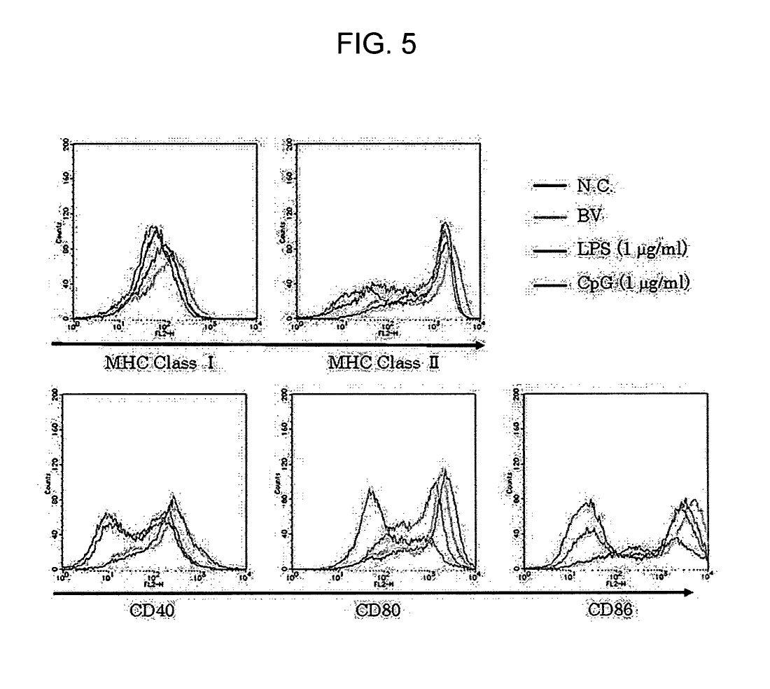

The dendritic cells (1.0×10⁶ cells), which had been exposed to the activating reagent containing 50 pfu of baculoviruses per dendritic cells in the exposure step (1), were used. As controls, untreated dendritic cells (N.C), and dendritic cells exposed to activating reagents containing LPS (1 μg/mL) or CpG-ODN (1 μg/mL) instead of the baculoviruses were used. The cells were incubated for 48 hours in the culturing step (3). Then, the dendritic cells were harvested, and the expressions of surface molecules were respectively measured by FACS. As shown in FIG. 5, the expression levels of the surface molecules of the dendritic cells remarkably increased by the incorporation of baculoviruses. The expression levels of the costimulator molecules, CD40, CD80 and CD86 remarkably increased compared to the levels of the costimulator molecules in the untreated dendritic cells. Therefore, it was considered that the dendritic cells were activated by the incorporation of baculoviruses and induced to be a mature state from an immature state.

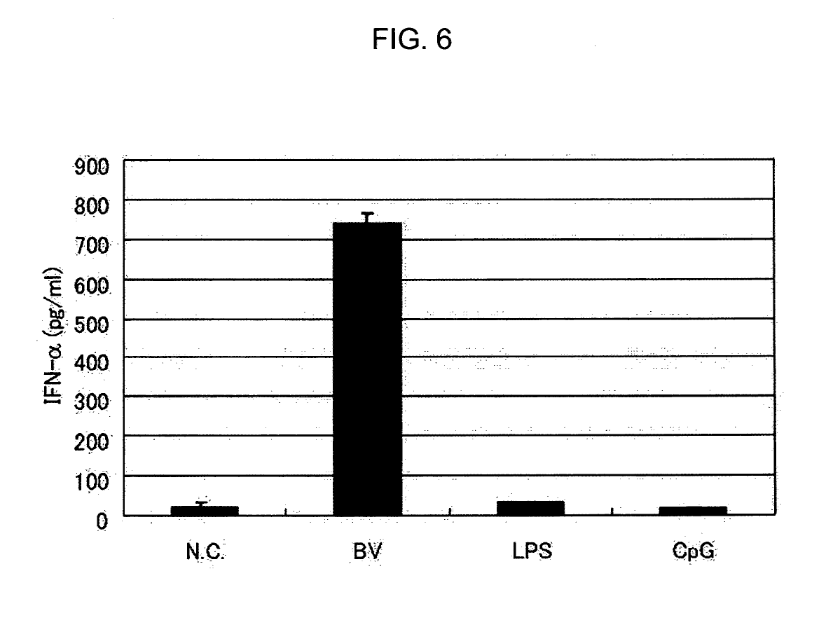

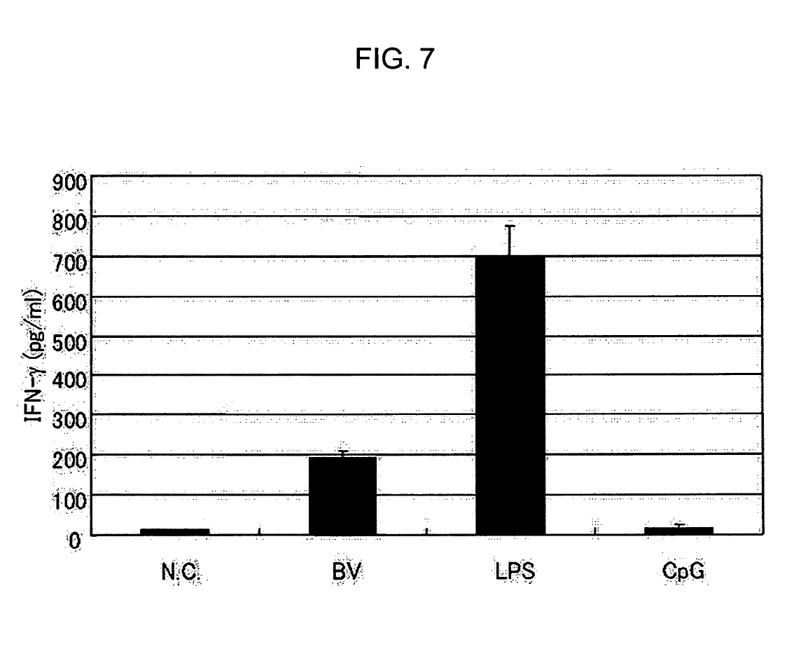

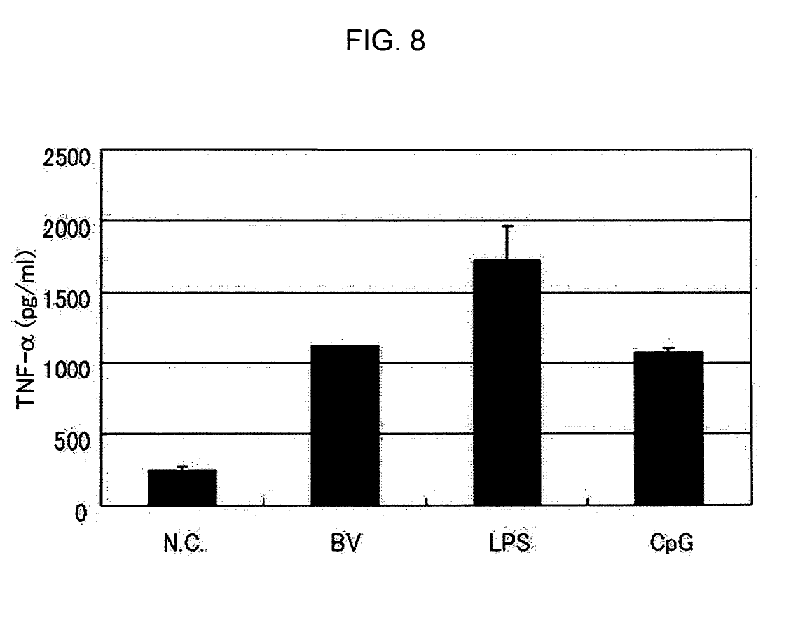

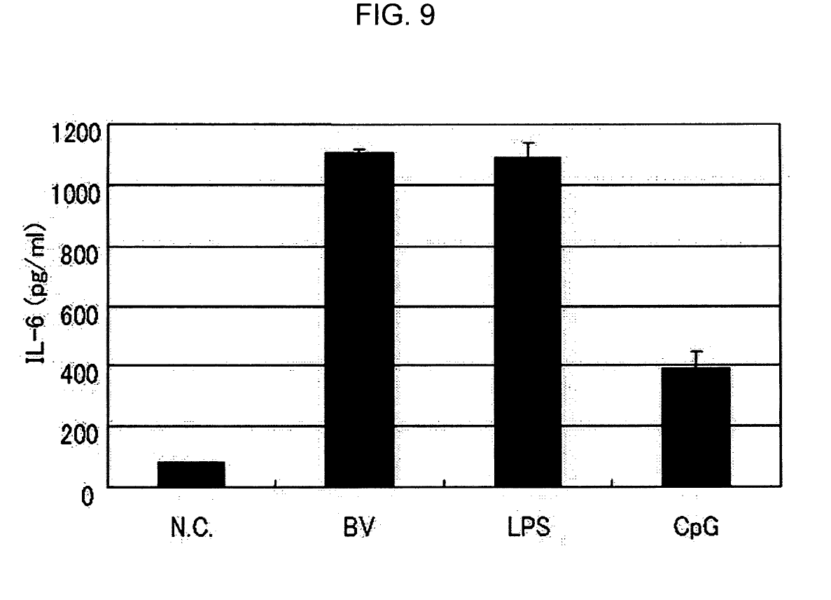

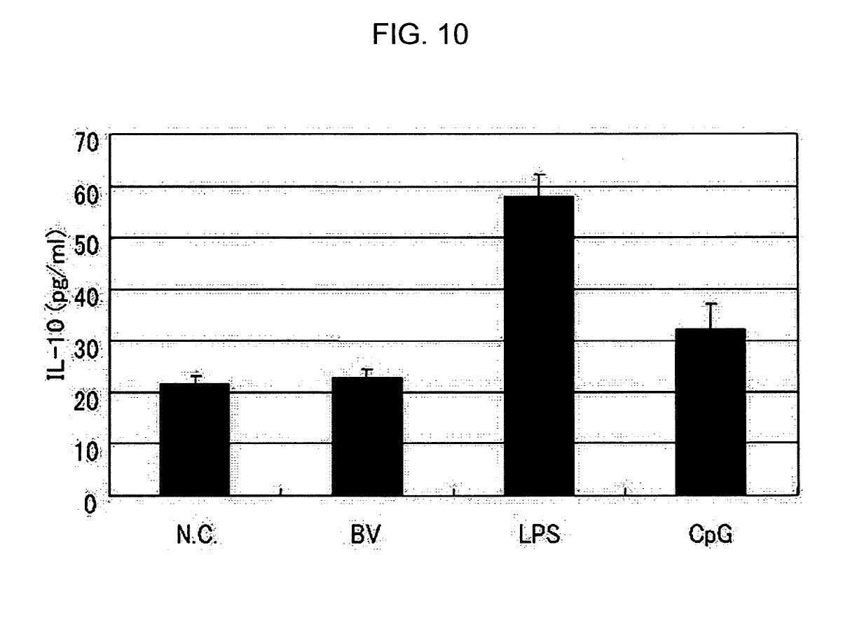

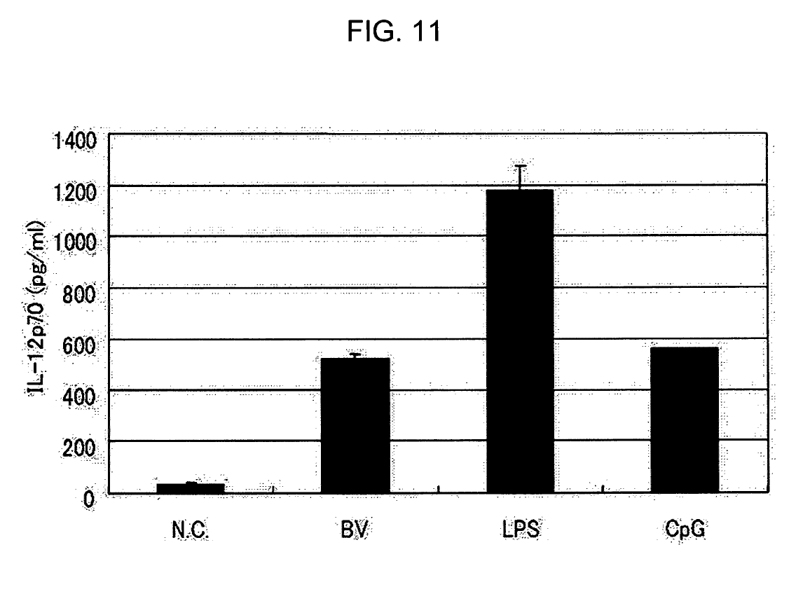

Furthermore, supernatant media were collected 48 hours after culturing. Then, the amounts of IFN-α, IFN-γ, TNF-α, IL-6, IL-10 and IL-12p70 produced were measured by ELISA (FIGS. 6-11). As shown in the figures, it was confirmed that the dendritic cells produced IFN-a and inflammatory cytokines (TNF-α, IL-6 and IL-12p70) by incorporating the baculoviruses. On the other hand, production of IL-10, a cytokine which inhibits T cells, was not observed. Accordingly, incorporation of baculoviruses by the dendritic cells elevated expressions of various cytokines, MHC molecules and costimulator molecules, and promoted an immune response.

<<3. Verification of Interactions between Baculovirus—Incorporated Dendritic Cells and Other Immune Cells>>

The dendritic cells that had incorporated the baculoviruses were co-cultured with NK cells and T cells. Then, it was studied if the dendritic cells would activate the NK cells and T cells.

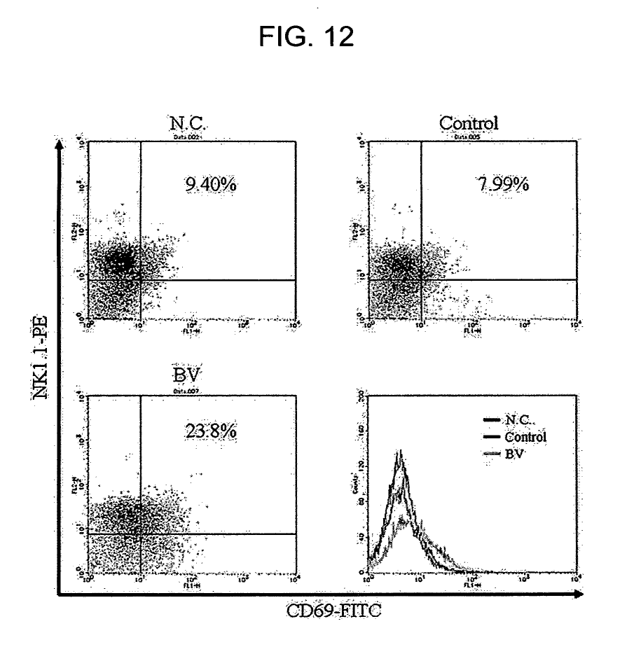

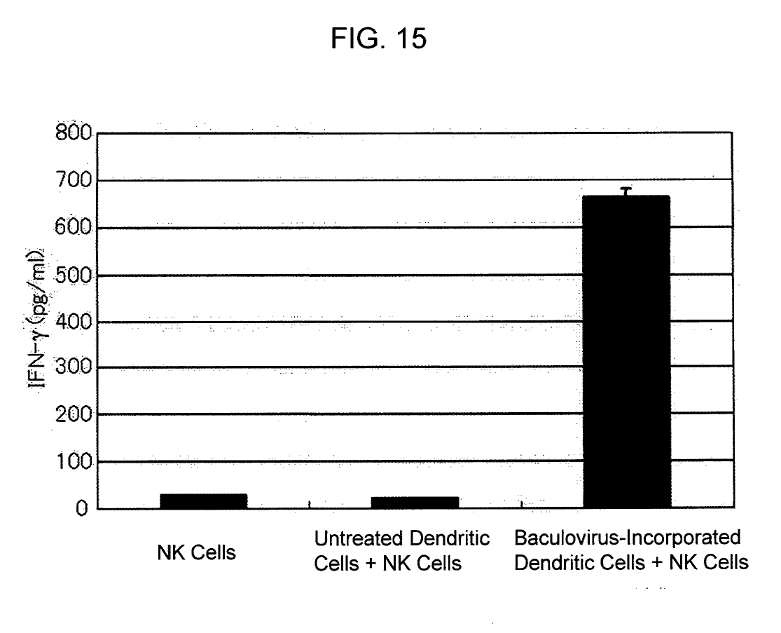

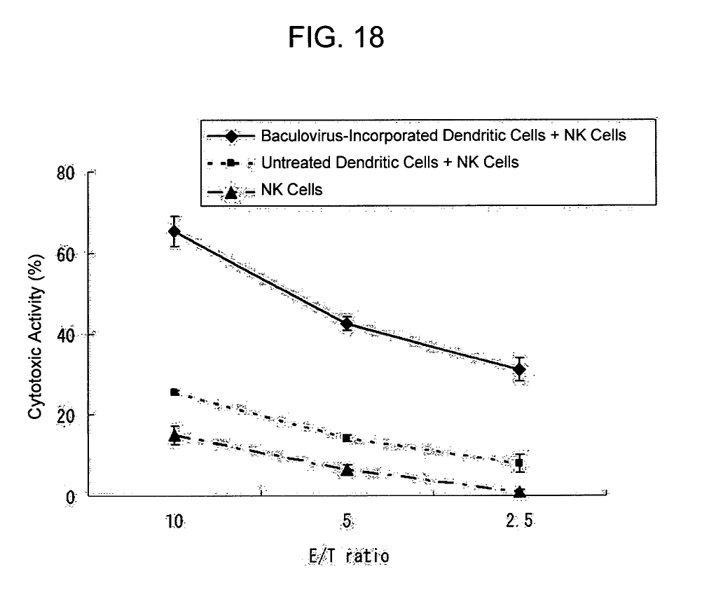

The dendritic cells (1.0×10⁶ cells), which had been exposed to the activating reagent containing 50 pfu of baculoviruses per dendritic cells in the exposure step (1), were co-cultured with NK cells obtained from mouse spleen for 18 hours. As a control, untreated dendritic cells were co-cultured with the NK cells. In the co-culture, the ratio of the number of dendritic cells to the number of NK cells was 1:2. After co-culturing, the cells were harvested. Then, the expression level of CD69, an early activation marker, was determined by FACS (FIG. 12). Also, the amount of IFN-γ in the medium supernatant was measured by ELISA (FIG. 15). Furthermore, cytotoxic activity of NK cells was also measured (FIG. 18).

As shown in FIG. 12, by co-culturing the NK cells with the dendritic cells that had incorporated the baculoviruses, the expression level of CD69 in the NK cells rose by approximately 2.5 times.

As shown in FIG. 15, by co-culturing the NK cells with the dendritic cells that had incorporated the baculoviruses, a large amount of IFN-γ was produced by the NK cells. It was considered that IL-12 produced by the baculovirus-incorporated dendritic cells activated the NK cells, and the activated NK cells produced the IFN-γ.

As shown in FIG. 18, by co-culturing the NK cells with the dendritic cells that had incorporated the baculoviruses, high cytotoxic activities of NK cells were observed at all the E/T ratios (ratio of effecter cells to tumor cells in the mixed culture).

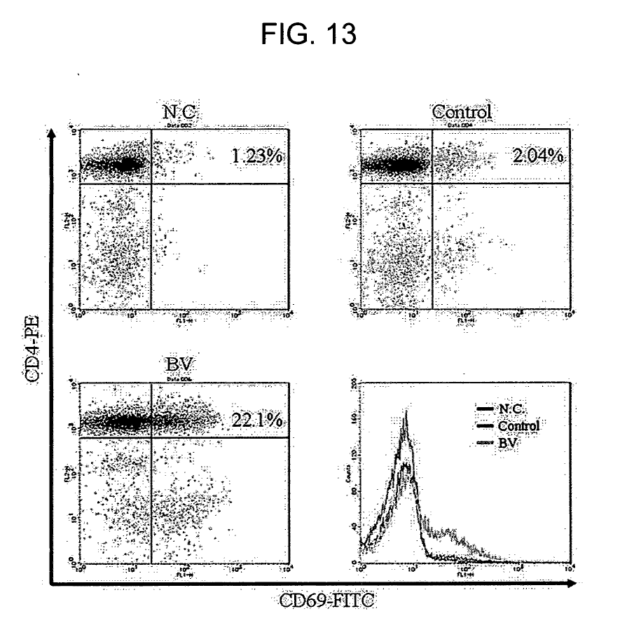

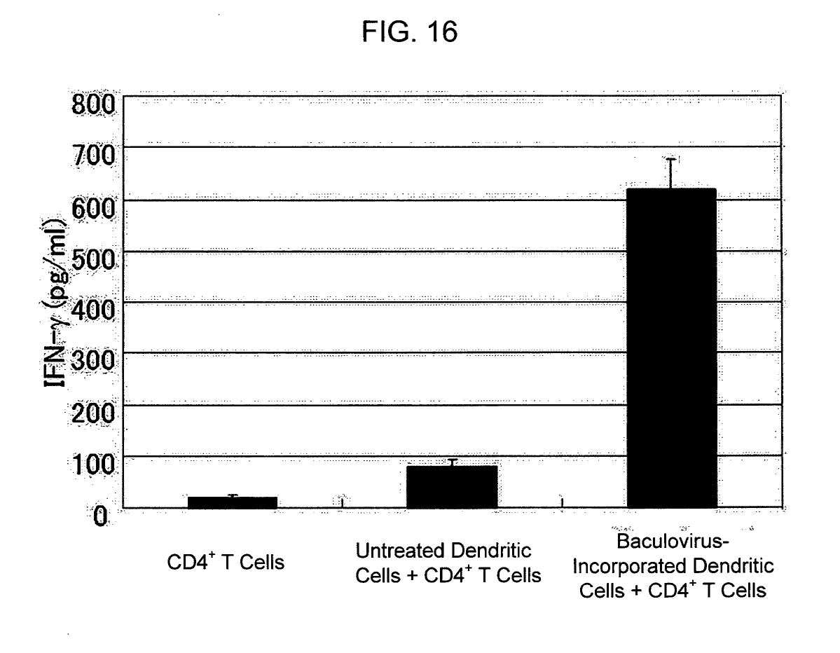

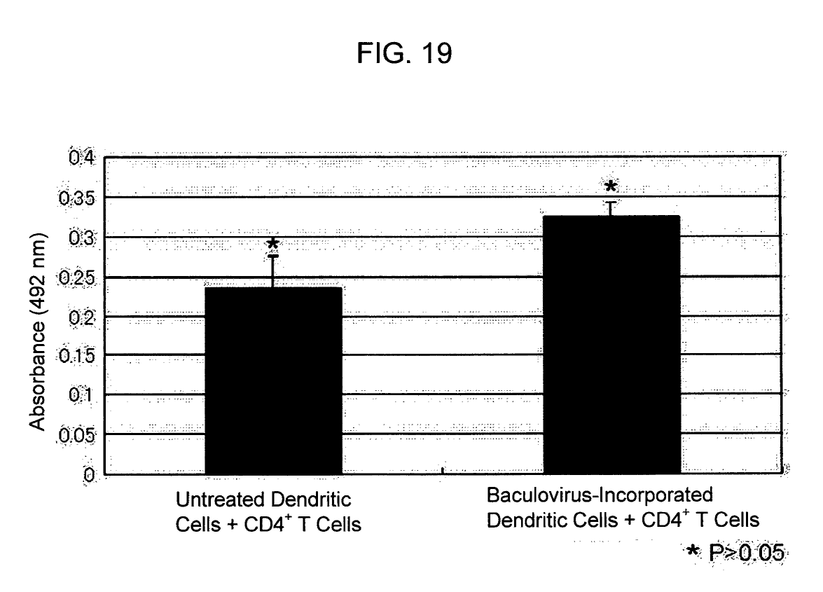

Furthermore, the dendritic cells (1.0×10⁶ cells), which had been exposed to the activating reagent containing 50 pfu of baculoviruses per dendritic cells in the exposure step (1), were co-cultured with CD4+ T cells and CD8+ T cells obtained from mouse spleen, respectively for 24 hours. As controls, untreated dendritic cells were co-cultured with the CD4+ T cells and CD8+ T cells respectively. In the co-culture, the ratios of the number of dendritic cells to the number of CD4⁴⁺ T cells and the number of CD8+ T were 1:10 respectively. After co-culturing, the cells were harvested. Then, the expression levels of CD69, an early activation marker, were determined by FACS (FIGS. 13 & 14). Also, the amounts of IFN-γ in the medium supernatant were measured by ELISA (FIGS. 16 & 17). Furthermore, proliferation activities of CD4+ T cells and CD8+ T cells were also measured (FIGS. 19 & 20).

As shown in FIGS. 13 & 14, by co-culturing the CD4+ T cells and CD8+ T cells with the dendritic cells that had incorporated the baculoviruses, the expression levels of CD69, which is an activation marker of CD4+ T cells and CD8+ T cells, rose by approximately 18 times in the CD4+ T cells and by approximately 32 times in the CD8+ T cells.

As shown in FIGS. 16 & 17, by co-culturing the CD4+ T cells and CD8+ T cells with the dendritic cells that had incorporated the baculoviruses, large amounts of IFN-γ were produced by the CD4+ T cells and CD8+ T cells respectively.

Absorbance of 492 nm light was measured to see the growth of T cells. As shown in FIGS. 19 & 20, the co-culture of baculovirus-incorporated dendritic cells activated the CD4+ T cells and CD8+ T cells and promoted the cell proliferations compared to the co-culture of untreated dendritic cells.

<<4. Verification of Antitumor Activity of Medical Composition Produced by the Method of Present Invention>>

The medical composition produced by the method of present invention (or just called ‘the medical composition’) was injected into tail veins of mice. Then, it was investigated if the medical composition induced immune responses of NK cells, CD4+ T cells and CD8+ T cells in the mouse spleen. Furthermore, the medical composition was administered to a lung cancer mouse model, and antitumor activities induced by the medical composition were investigated.

Verification of Immune Response Induced by the Medical Composition The dendritic cells (1.0×10⁶ cells) were exposed to the activating reagent containing 50 pfu of baculoviruses per dendritic cells in the exposure step (1). Then, the dendritic cells were incubated for 6 hours in the culturing step (3), and suspended in 100 μL of PBS. Thereby, the medical composition was produced (Example 1). As comparative examples, the following medical compositions were prepared: a medical composition containing dendritic cells prepared by culturing for 6 hours without carrying out the exposure step (1) (Comparative Example 1); a medical composition containing dendritic cells stimulated by 1 μg/mL of LPS and cultured for 6 hours (Comparative Example 2); and a medical composition containing dendritic cells stimulated by 1 μg/mL of CpG-ODN and cultured for 6 hours (Comparative Example 3).

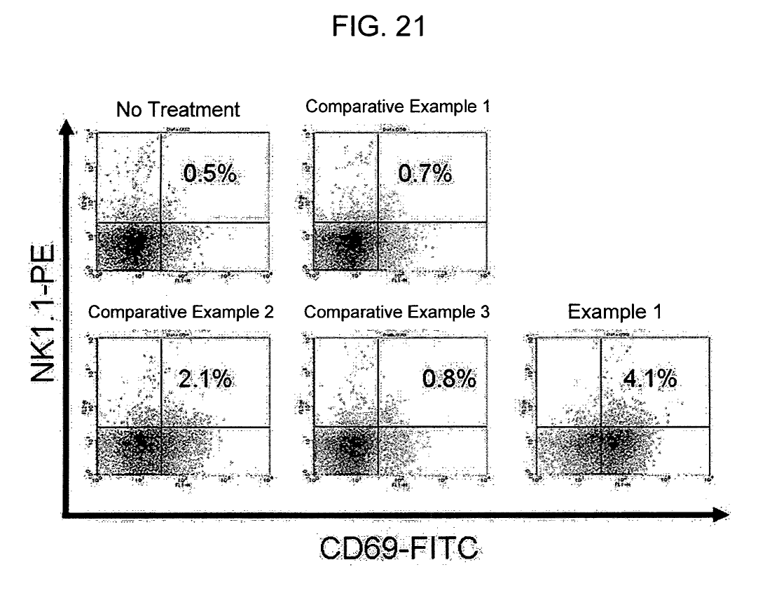

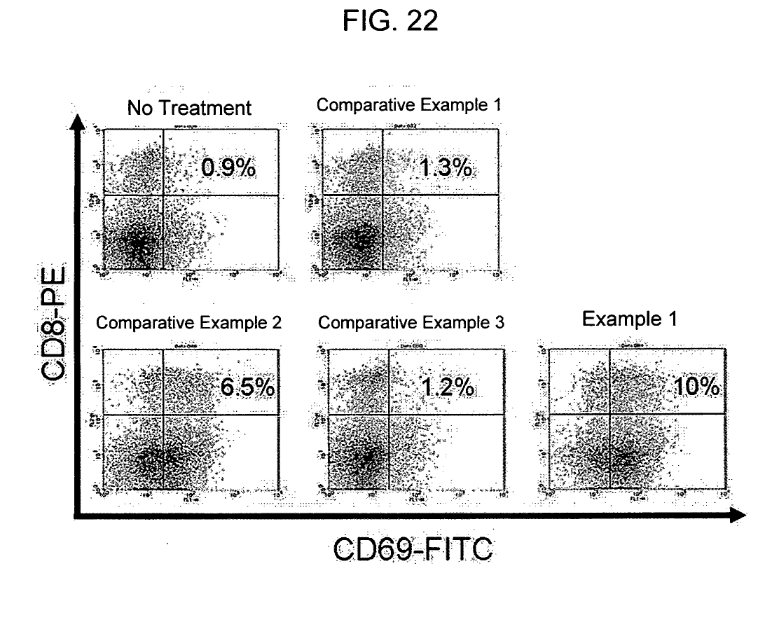

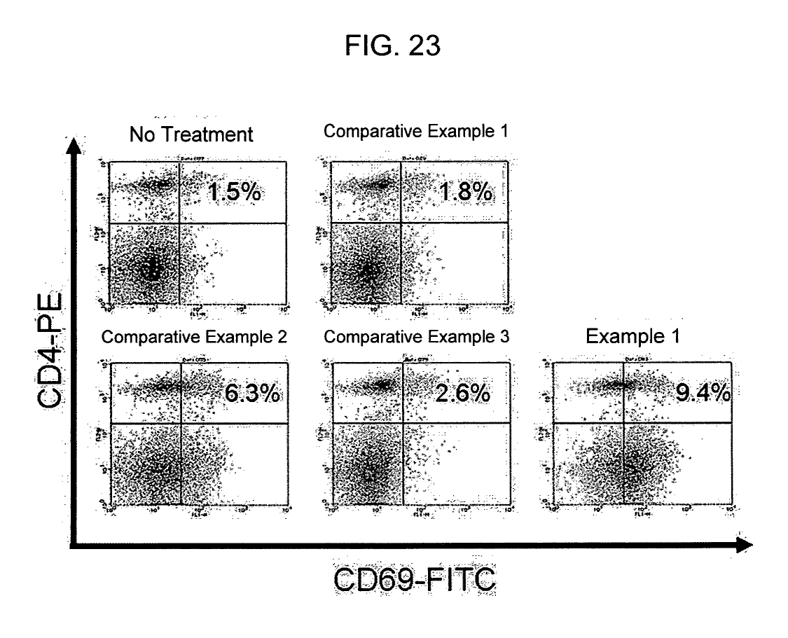

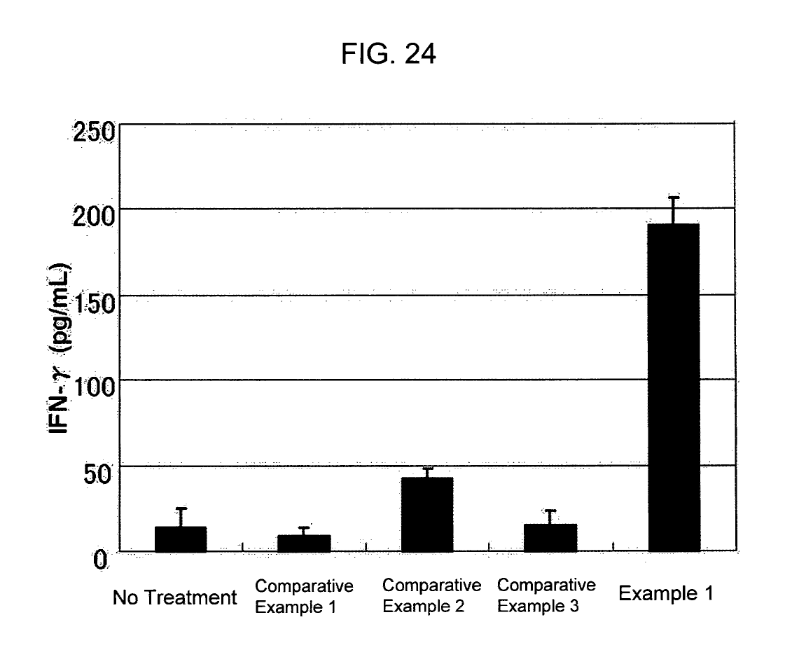

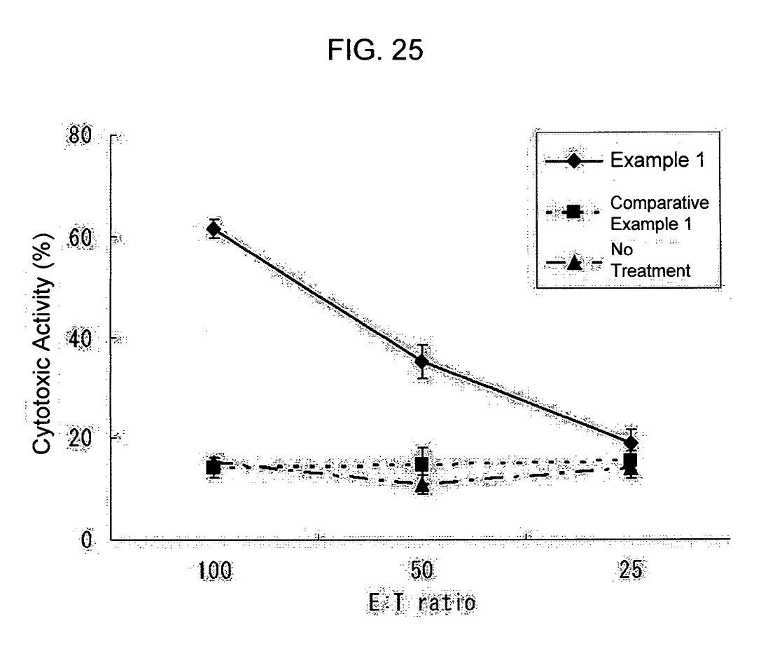

The medical compositions of Example 1 as well as Comparative Examples 1-3 were injected into mouse tail veins (n=3). 6 hours later, their spleens were dissected out, and at the same time bloods were collected from their hearts. Spleen cells were double-stained with an antibody for CD69, which is the activation marker of immune cells, and antibodies for NK1.1, CD4 and CD8, which are the markers of NK cells, CD4+ T cells and CD8+ T cells respectively. Then, the expression levels of these markers were measured by FACS (FIGS. 21-23). Furthermore, serums were obtained by centrifuging the bloods, and the amounts of IFN-γ in the serums were measured by ELISA (FIG. 24). Moreover, cytotoxic activities of the spleen cells were measured (FIG. 25).

As shown in FIGS. 21-23, by injecting the medical composition of Example 1 into the tail vein, the expression levels of CD69, which is the activity marker of NK cells, CD4+ T cells and CD8+ T cells in the spleen, increased. The expression levels were approximately 8.2 times higher in the NK cells, approximately 6.3 times higher in the CD4+ T cells and approximately 11 times higher in the CD8+ T cells than those of the untreated cells. Furthermore, as shown in FIG. 24, it was confirmed that the injection of the medical composition of Example 1 into the tail vein induced the production of IFN-γ in the blood. Moreover, as shown in FIG. 25, the NK cells and cytotoxic T cells (CD8+ T cells) were activated by the medical composition of Example 1, and their cytotoxic activities increased.

To verify the antitumor activity of the medical composition, a lung cancer mouse model was generated.

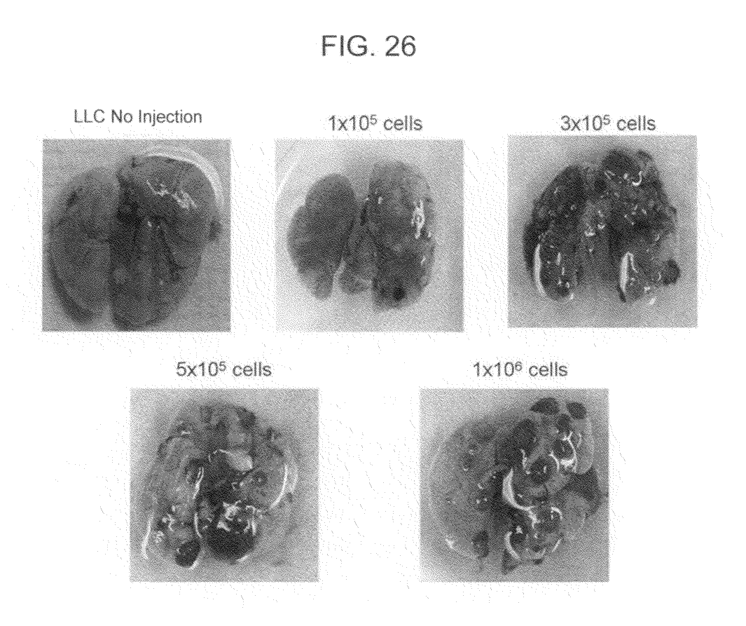

Generation of Lung Cancer Mouse Model 1×10⁵ cells, 3×10⁵ cells, 5×10⁵ cells, and 1×10⁶ cells of LLC were injected into the tail veins of C57BL/6 mice. 28 days later, the mice were dissected and the pathologies of lungs were observed. As shown in FIG. 26, much more prominent tumors were formed in the mice to which 1×10⁶ cells of LLC were injected than the mice to which other numbers of cells were injected. Therefore, in the following experiments, the lung cancer mouse model generated by injecting 1×10⁶ cells of LLC was used.

Verification of Antitumor Effect of the Medical Composition

The medical compositions of Example 1, Comparative Example 1 and Comparative Example 3 were injected into tail veins of the lung cancer mouse model. Administration of the medical composition was either given once (the 3rd day after LLC administration) or twice (once on the 3rd day after LLC administration and once on the 6th day after LLC administration). 28 days after administering the LLC, the mice were dissected. Then, the numbers of lung nodules were counted, and lung tissues were pathologically observed and histologically analyzed by HE staining. Furthermore, survival rates were measured on the lung cancer mouse model, to which the medical composition was administered once.

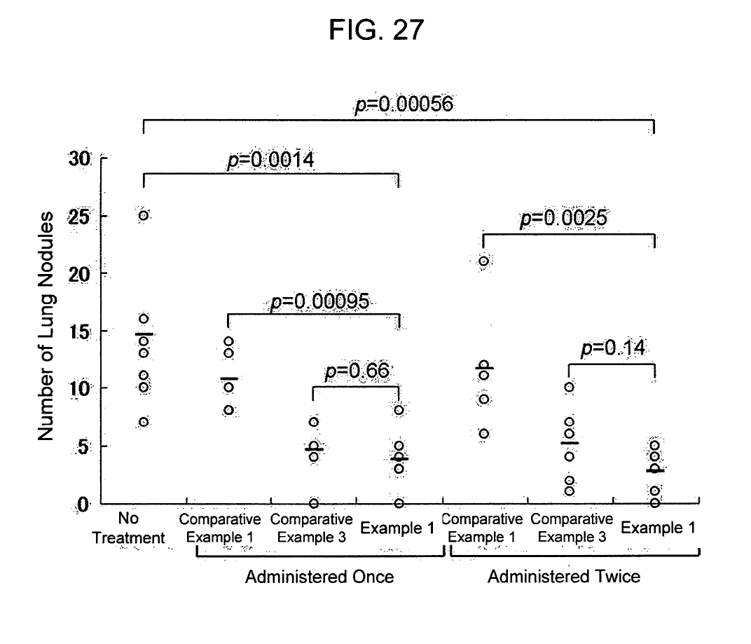

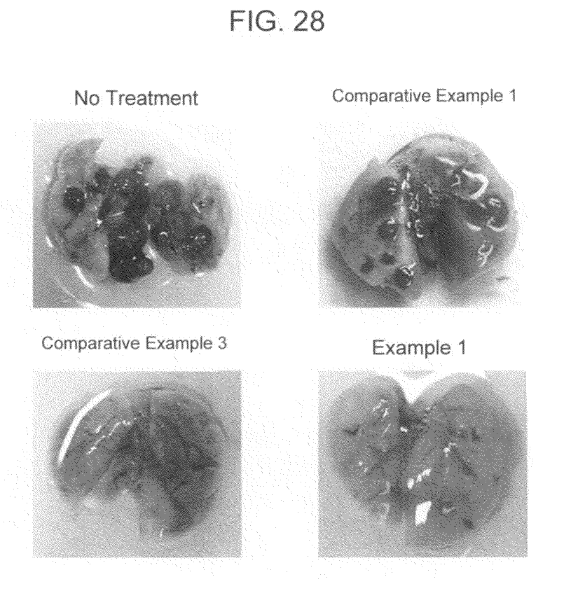

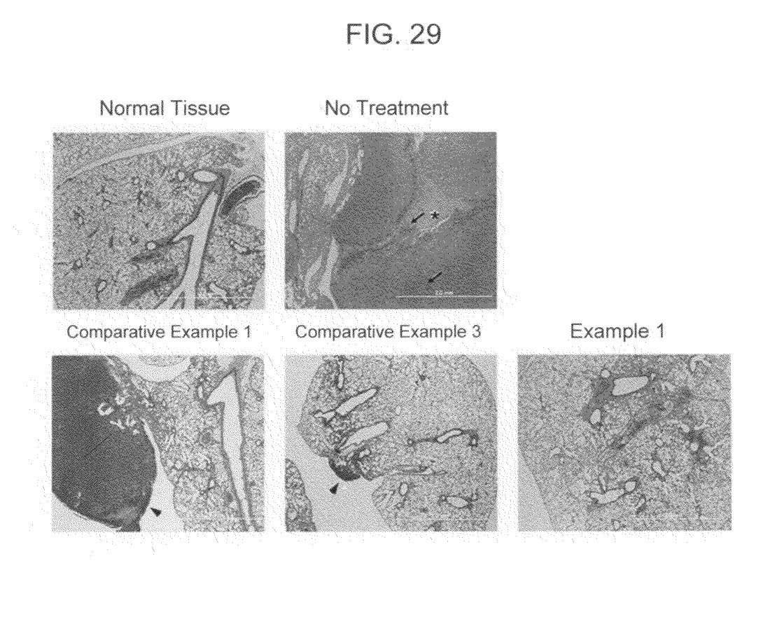

As shown in FIG. 27, superior suppression of tumor formation was observed in the mice treated with the medical composition of Example 1 compared to the untreated mice and the mice treated with the medical composition of Comparative Example 1. Even from the photographs of lung tissues shown in FIG. 28, suppression of tumor formation was evident in the mice treated with the medical composition of Example 1. As shown in FIG. 29, the histological analysis using HE staining on lung revealed tumor formation, bleeding and tissue necrosis in the untreated mice and the mice treated with the medical compositions of Comparative

Examples. On the other hand, tumor formation was suppressed and the tissue morphology was almost the same as normal tissue of mice to which the medical composition of Example 1 was administered.

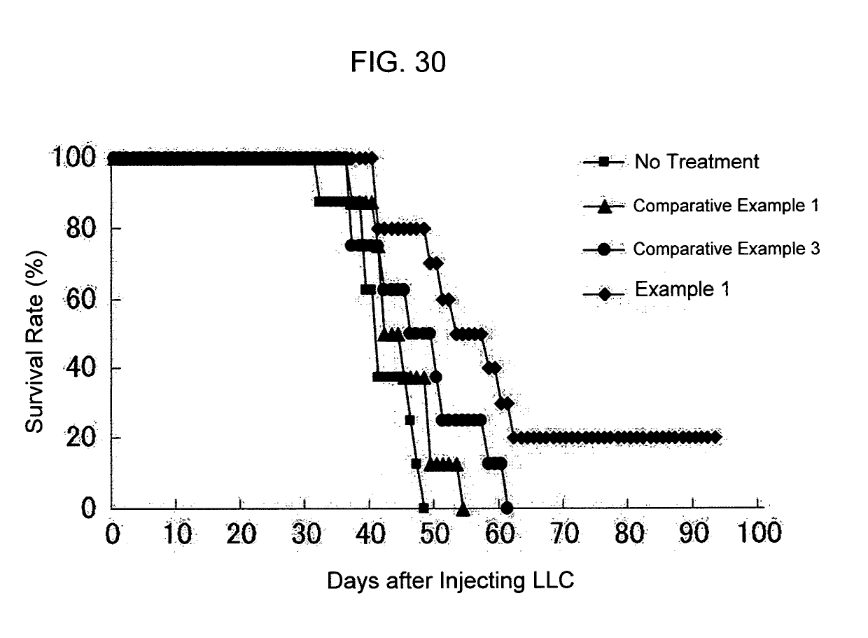

Also, survival rates of the lung cancer mouse model were measured on the mice to which the medical composition was administered once. As shown in FIG. 30, while all the mice of Comparative Example 1 and Comparative Example 3 died by day 60, the mice treated with the medical composition of Example 1 showed a significantly higher survival rate than those of the Comparative Examples.

As described above, administration of the medical composition of the present invention suppressed tumor formation and brought a significant therapeutic effect.

### INDUSTRIAL APPLICABILITY

The medical composition produced by the method of present invention is suitably applied to cancer therapy and/or cancer prevention.

1-7. (canceled)

8. A method of producing a medical composition comprising an antigen-presenting cell having an antitumor activity, said method comprising the steps of:
exposing an antigen-presenting cell to an activating reagent comprising a baculovirus; and
separating the antigen-presenting cell from the activating reagent.

9. The method of claim 8, further comprising the step of: incubating the antigen-presenting cell after separating the antigen-presenting cell from the activating reagent.

10. The method of claim 8, further comprising the step of: checking an absence of the baculovirus in a solution comprising the antigen-presenting cell after separating the antigen-presenting cell from the activating reagent.

11. The method of claim 8, wherein the activating reagent comprises approximately 50 pfu of the baculovirus per antigen-presenting cell.

12. The method of claim 8, wherein the antigen-presenting cell originates from a cancer patient.

13. The method of claim 8, wherein the antigen-presenting cell is a dendritic cell.

14. The method of claim 8, wherein the baculovirus is absent in the medical composition.

15. The method of claim 8, wherein said antigen-presenting cell is exposed with at least 10 pfu of said activating reagent per antigen-presenting cell and at most 100 pfu of said activating reagent per antigen-presenting cell.

16. The method of claim 8, wherein said antigen-presenting cell is exposed to said activating reagent for at least 30 minutes and at most 2 hours; and
wherein the activating reagent is stirred at least once while the antigen-presenting cell is exposed to the activating reagent.

17. The method of claim 9, wherein said incubation is at least 1 hour and at most 48 hours with an incubation temperature of at least 30° C. and at most 40° C.

18. The method of claim 8, further comprising the step of: testing for the presence of baculovirus in a solution comprising the antigen-presenting cell using polymerase chain reaction or immunostaining after separating the antigen-presenting cell from the activating reagent.

19. The method of claim 8, further comprising the step of: washing the antigen-presenting cell after separating the antigen-presenting cell from the activating reagent.

20. The method of claim 19, further comprising the step of: repeating washing the antigen-presenting cell until the baculovirus is not detected in a solution comprising the antigen-presenting cell.

21. The method of claim 8, further comprising the step of: adjusting a volume of the activating reagent after exposing the antigen-presenting cell to the activating reagent and before separating the antigen-presenting cell from the activating reagent.

22. The method of claim 8, wherein the activating reagent during separation of the antigen-presenting cell has a lower temperature than the activating reagent during exposure of the antigen-presenting cell to the activating reagent.

23. The method of claim 8, wherein the baculovirus is a wild-type baculovirus.

24. The method of claim 8, wherein the medical composition contains at most 1×10⁶ of the antigen-presenting cells per 100 μL of the medical composition.

25. The method of claim 8, further comprising the step of: exposing the antigen-presenting cell to the activating reagent so that expression levels of IFN-α, TNF-α, IL-6 and IL-12p70 in the antigen-presenting cell 48 hours after the exposure are higher than those in a control antigen-presenting cell, which has been exposed to a control reagent lacking the baculovirus under equivalent condition as was done for the antigen-presenting cell.

26. A method of treating a cancer or tumor; the method comprising the steps of:
preparing an antigen-presenting cell;
exposing the antigen-presenting cell to a baculovirus;
removing the baculovirus that has not been uptaken by the antigen-presenting cell; and
administering the antigen-presenting cell to a patient or an animal.

27. The method of claim 26, further comprising the step of: taking out an antigen-presenting cell from a patient or an animal to prepare the antigen-presenting cell.
